(19) United States
(12) Patent Application Publication
(10) Pub. No.: US 2025/0359876 A1
(43) Pub. Date: Nov. 27, 2025

(54) INSTRUMENTS AND SURGICAL METHODS FOR BUNION PROCEDURES

(71) Applicant: Paragon 28, Inc., Englewood, CO (US)

(72) Inventors: Albert DACOSTA, Lone Tree, CO (US); Michael SCHMIDT, Greenwood Village, CO (US); Garrett Jeffrey LIPKER, Arvada, CO (US); Lucas Charles MCMAHAN, Lone Tree, CO (US); Kenneth Allan ROGGOW, Denver, CO (US); Benjamin MAJORS, Englewood, CO (US); Jason Allan EDIE, Sandy, UT (US)

(73) Assignee: Paragon 28, Inc., Englewood, CO (US)

(21) Appl. No.: 19/290,755

(22) Filed: Aug. 5, 2025

Related U.S. Application Data
(63) Continuation of application No. PCT/US2024/014946, filed on Feb. 8, 2024.
(60) Provisional application No. 63/483,885, filed on Feb. 8, 2023.

(51) Int. Cl. A61B 17/17; A61F 2/42; A61F 2/46

(52) U.S. Cl. CPC A61B 17/1775; A61F 2/4225; A61F 2/4606; A61F 2002/4238

(57) ABSTRACT

An instrument system includes a first cut guide and an actuation instrument. The actuation instrument includes an upper portion having a handle and a first cannulation, and a lower portion releasably couplable with the upper portion. The lower portion includes a shaft, a second cannulation, wherein the second cannulation is coaxial and in fluid communication with the first cannulation, and a paddle configured to abut a resected surface of a first bone. The instrument system also includes a compression mechanism releasably couplable with the lower portion, the compression assembly couplable with the first bone and a second bone The compression assembly includes an actuator configured to bias at least one of the first bone and the second bone toward the other of the first bone and the second bone. A method for using the instrument system is also disclosed herein.

### CROSS REFERENCE TO RELATED APPLICATIONS

This application is a bypass continuation of International Patent Application PCT/US2024/014946, filed Feb. 8, 2024, and entitled “Instruments and Surgical Methods For Bunion Procedures,” which claims the benefit of priority of U.S. Provisional Patent Application No. 63/483,885, filed on Feb. 8, 2023, and entitled “Instruments and Surgical Methods For Bunion Procedures”, the disclosures of each of these applications are hereby incorporated herein by reference in their entirety.

### TECHNICAL FIELD

The present disclosure relates to surgical instruments, guides, and methods of use to be implemented in surgical procedures. The present disclosure relates to podiatric and orthopedic surgical instruments, guides, and methodology to be implemented in various procedures of the foot and/or ankle, for example arthrodesis. More specifically, but not exclusively, the present disclosure relates to surgical instruments, guides to be implemented in conjunction with instruments (as well as other components, for example implants, devices, systems, assemblies, etc.) and methods of use for performing procedures to address bunions.

### BACKGROUND OF THE INVENTION

Many currently available surgical instruments and guides, as well as surgical methods, do not completely address the needs of patients. Additionally, many currently available surgical instruments, guides, and surgical methods fail to account for properties of joint anatomy and accordingly can decrease the probability of favorable outcomes for the patient.

### SUMMARY OF THE INVENTION

The present disclosure is directed toward implants and implant systems for procedures involving the foot and/or ankle. More specifically, the present disclosure is directed to implants and implant systems for ankle procedures.

One aspect of the present disclosure is directed to an instrument system. The instrument system includes a first cut guide and an actuation instrument. The actuation instrument includes an upper portion having a handle and a first cannulation, and a lower portion releasably couplable with the upper portion. The lower portion includes a shaft, a second cannulation, wherein the second cannulation is coaxial and in fluid communication with the first cannulation, and a paddle configured to abut a resected surface of a first bone. The instrument system also includes a compression mechanism releasably couplable with the lower portion, the compression assembly is couplable with the first bone and a second bone. The compression assembly includes an actuator configured to bias at least one of the first bone and the second bone toward the other of the first bone and the second bone.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front, medial, perspective view of an instrument system, in accordance with the present disclosure;

FIG. 2 is a front, lateral, perspective view of the instrument system of FIG. 1, in accordance with the present disclosure;

FIG. 3 is a top view of the implant system of FIG. 1, in accordance with the present disclosure;

FIG. 4 is a front view of the instrument system of FIG. 1, in accordance with the present disclosure;

FIG. 5 is a medial view of the instrument system of FIG. 1, in accordance with the present disclosure;

FIG. 6 is a lateral view of the instrument system of FIG. 1, in accordance with the present disclosure;

FIG. 7A is a first side perspective view of a component of the instrument system of FIG. 1, in accordance with the present disclosure;

FIG. 7B is a second side perspective view of a component of the instrument system of FIG. 1, in accordance with the present disclosure;

FIG. 8 is a medial perspective view of an exemplary configuration of the components of the instrument system of FIG. 1, in accordance with the present disclosure;

FIG. 9 is a top view of the exemplary configuration of FIG. 8 of the components of the instrument system of FIG. 1, in accordance with the present disclosure;

FIG. 10 is a medial view of the exemplary configuration of FIG. 8 of the components of the instrument system of FIG. 1, in accordance with the present disclosure;

FIG. 11 is a top view of the exemplary configuration of FIG. 8 of the components of the instrument system of FIG. 1, in accordance with the present disclosure;

FIG. 12 is a medial view of an exemplary configuration of the components of the instrument system of FIG. 1, in accordance with the present disclosure;

FIG. 13 is a top view of the exemplary configuration of FIG. 12 of the component of the instrument system of FIG. 1, in accordance with the present disclosure;

FIG. 14 is a flow chart of an exemplary method in which the instrument system of FIG. 1 may be implemented, in accordance with the present disclosure;

FIG. 15 is a medial perspective view of an instrument system, in accordance with the present disclosure;

FIG. 16 is a lateral perspective view of the instrument system of FIG. 15, in accordance with the present disclosure;

FIG. 17 is an enlarged top perspective view of the instrument system of FIG. 15, in accordance with the present disclosure;

FIG. 18 is a rear view of the instrument system of FIG. 15, in accordance with the present disclosure;

FIG. 19A is an enlarged medial side view of the instrument system of FIG. 15, in accordance with the present disclosure;

FIG. 19B is an enlarged view of the instrument system of FIG. 15, in accordance with the present disclosure;

FIG. 20 is an enlarged top view of the instrument system of FIG. 15, in accordance with the present disclosure;

FIG. 21 is an enlarged medial side view of the instrument system of FIG. 15, in accordance with the present disclosure; and

FIG. 22 is a flow chart of an exemplary method in which the instrument system of FIG. 15 may be implemented, in accordance with the present disclosure.

### DETAILED DESCRIPTION OF THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in International PCT Application No. PCT/US2018/20046, filed on Feb. 27, 2018, and entitled Intramedullary Nail Alignment Guides, Fixation Guides, Devices, Systems, and Methods of Use; International PCT Application No. PCT/US2018/64368, filed on Dec. 17, 2018, and entitled Alignment Guides, Cut Guides, Systems and Methods of Use and Assembly; International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or U.S. Pat. No. D865,173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or U.S. Pat. No. D9,042,010 filed on Apr. 9, 2019 and entitled Cut Guide; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in U.S. Provisional Application No. 63/173,043, filed Apr. 9, 2021 and entitled Surgical Instruments, Guides, and Methods of Use; and/or International PCT Application No. PCT/US2018/20046, filed on Feb. 27, 2018, and entitled Intramedullary Nail Alignment Guides, Fixation Guides, Devices, Systems, and Methods of Use; and/or International PCT Application No. PCT/US2018/64368, filed on Dec. 17, 2018, and entitled Alignment Guides, Cut Guides, Systems and Methods of Use and Assembly; and/or International PCT Application No. PCT/US2019/041146, filed on Jul. 10, 2019, and entitled Guides, Instruments, Systems and Methods of Use; and/or International PCT Application No. PCT/US2014/27086, filed on Mar. 14, 2014, and entitled Intramedullary Nail Fixation Guides, Devices, and Methods of Use; and/or U.S. Pat. No. 9,980,760 filed on Nov. 19, 2014, and entitled Step Off Bone Plates, Systems, and Methods of Use; and/or U.S. Pat. No. D720,456 filed on Jul. 26, 2012 and entitled Lapidus Bone Wedge; and/or U.S. Pat. No. D765,844 filed on Oct. 23, 2014 and entitled Bone Plate; and/or U.S. Pat. No. D695,402 filed on Dec. 10, 2013 and entitled Lapidus Cut Guide; and/or U.S. Pat. No. D904,2016 filed on Nov. 22, 2017 and entitled Intramedullary Fastener; and/or United States Patent No. D865, 173 filed on Jul. 9, 2018 and entitled Cut Guide; and/or U.S. patent application Ser. No. 29/686,941 filed on Apr. 9, 2019 and entitled Cut Guide; and/or U.S. Pat. No. D904,609 filed on Apr. 9, 2019 and entitled Cut Guide; and/or U.S. Pat. No. D9042010 filed on Apr. 9, 2019 and entitled Cut Guide; and/or U.S. Provisional Patent Application No. 63/262,845 filed on Oct. 21, 2021 and entitled Surgical Instruments, Guides, and Methods of Use; and/or U.S. Provisional Patent Application No. 63/304,144 filed on Jan. 28, 2022 and entitled Surgical Instruments, Guides, and Methods of Use; which are hereby incorporated herein by reference in their entireties.

Procedures to address deformities such as bunions and anatomical structures of and around the Lapidus joint frequently require the positioning/repositioning and/or rotation/derotation of the first metatarsal. Referred to herein as the “Lapidus” joint, this joint may also be known and referred to as the first tarsometatarsal joint. It is common for a procedure on the Lapidus joint (e.g., fusion/arthrodesis) to require that the first metatarsal be manipulated by applying one or more forces to the first metatarsal. In some procedures, this manipulation is necessary before any cutting and/or preparation and subsequent fusion of the Lapidus joint can take place. In evaluating a Lapidus joint deformity, two different criteria are typically analyzed for correction. One of these criteria is the intramedullary angle formed between the longitudinal axes of the first metatarsal and the second metatarsal. Bunion deformities and other conditions of the Lapidus joint often include the first metatarsal shifting medially from a normal anatomical position, thus increasing the IM angle between the first and second metatarsals from what can be considered an anatomically correct range of angle measures. Rotation of the first metatarsal is also analyzed, as bunion deformities and other conditions of the Lapidus joint commonly include a first metatarsal that has rotated substantially in the frontal plane in a substantially clockwise direction (when viewed from an anterior to posterior direction). Commonly, a Lapidus joint procedure such as those mentioned previously requires manipulation of the first metatarsal so as to: a) correct (e.g., decrease) the IM angle between the first and second metatarsals by applying a substantially lateral force to the first metatarsal; and/or b) derotate the first metatarsal which as rotated from a normal anatomical position by applying a rotational force in a substantially counterclockwise direction when the first metatarsal is viewed in an anterior to posterior direction.

Referring to the drawings included herein, instrument systems and associated methods are shown and described. It should be understood that one or more of the instrument systems and/or associated methods shown and described herein may be implemented in conjunction with one or more of the other various instrument systems, components thereof, and associated methods shown and described herein. Further, it should be understood that the instrument systems and methods shown herein-as well as components thereof-may be duplicated, eliminated, or otherwise combined/modified and incorporated in conjunction with the same or other systems including but not limited to those shown and described herein and those incorporated by reference previously herein.

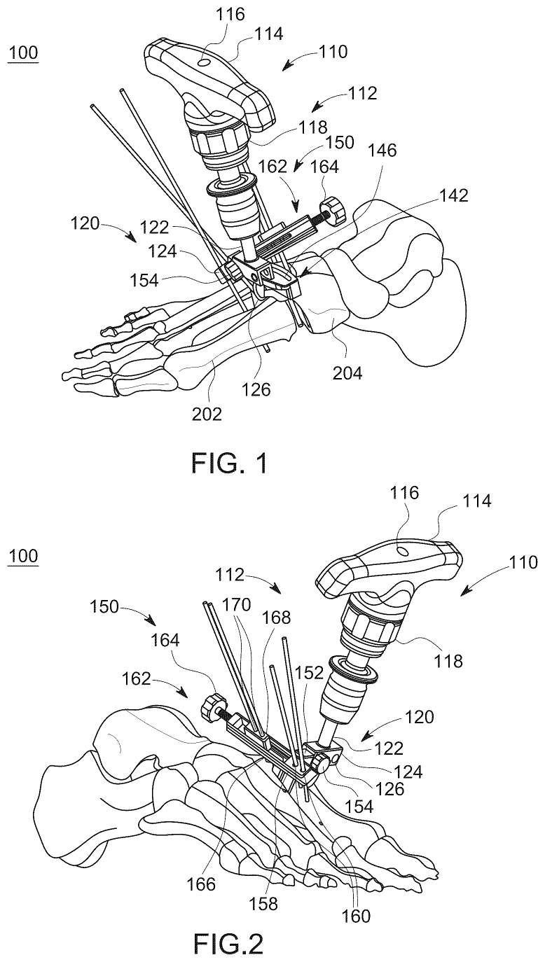

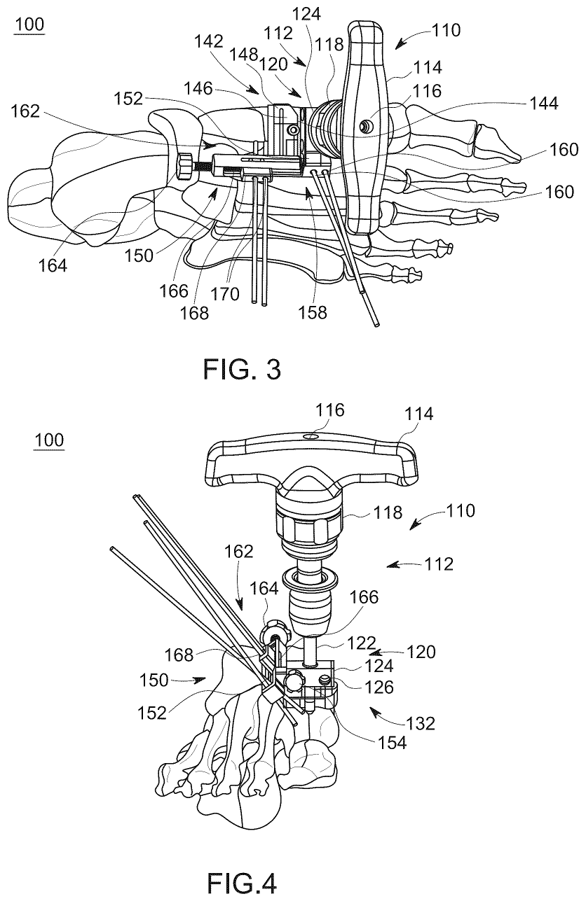

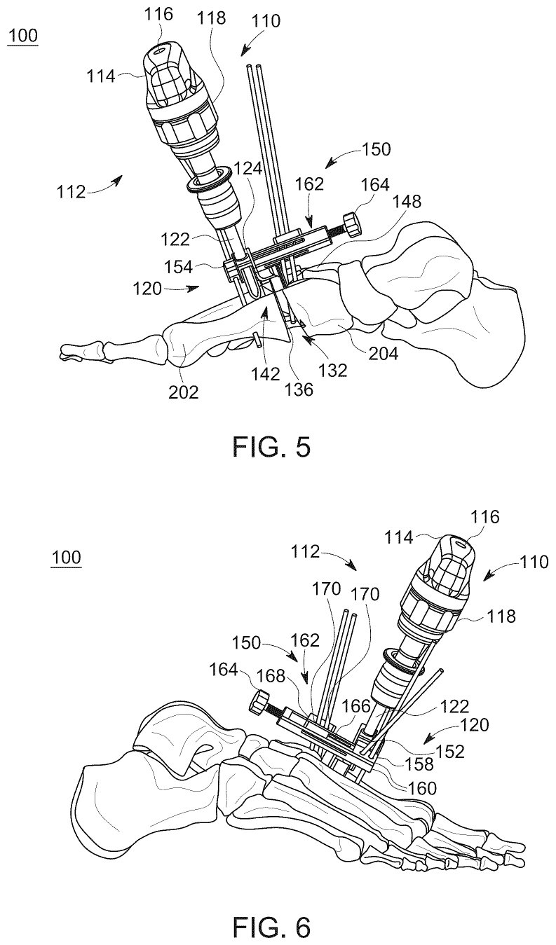

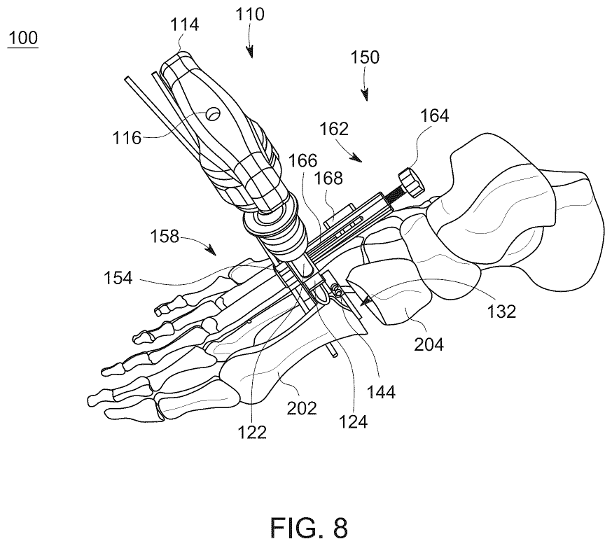

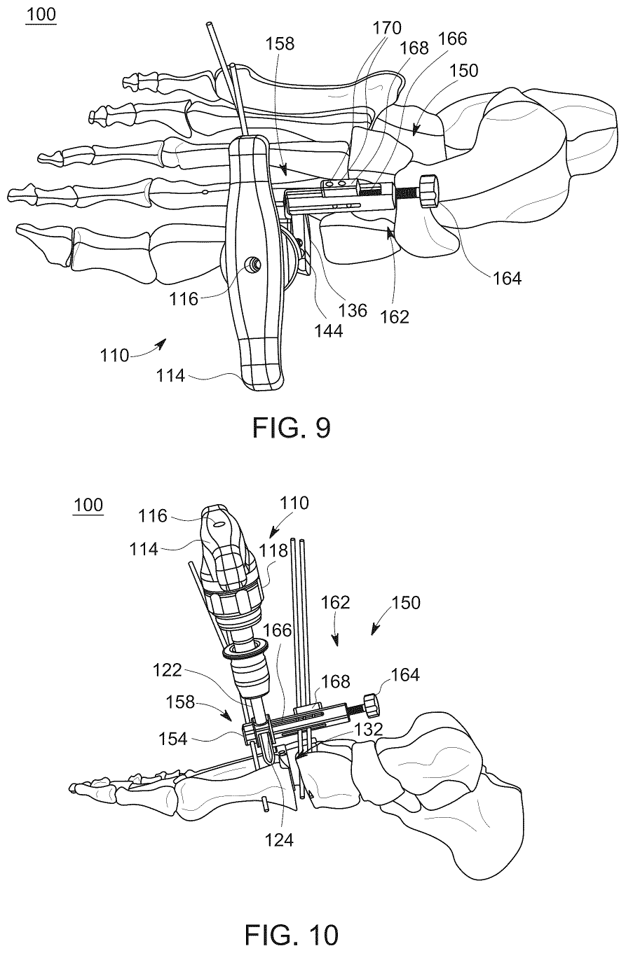

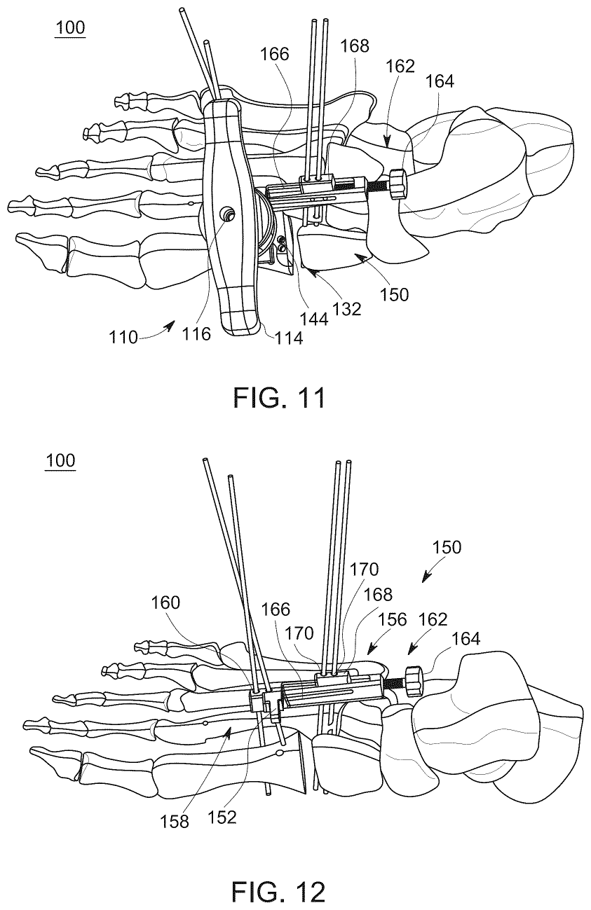

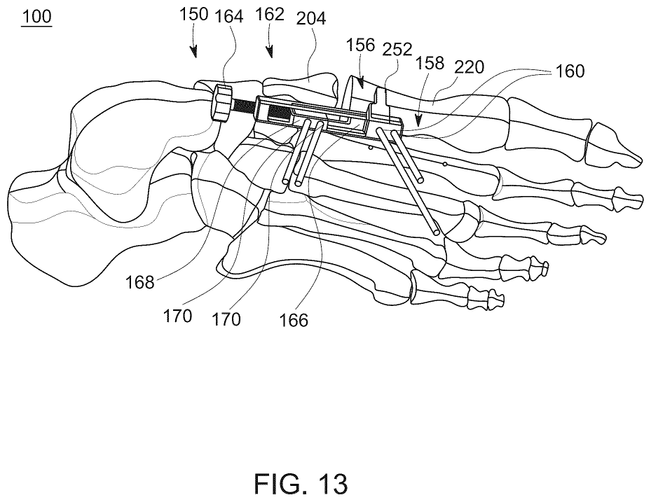

Referring now to FIGS. 1-13, an actuation instrument system 100 (referred to hereinafter as “system 100”) is shown, according to an exemplary embodiment. The system 100 may be implemented in conjunction with various additional instrumentation including those that are common to orthopedic procedures, for example cutting instruments and drills. Further, the system 100 may also be implemented in conjunction with various implant systems and associated instrumentation including, for example, intramedullary nails, bone plates, bone fasteners (e.g., screws, staples, etc.) and instrumentation configured to facilitate the implantation of the aforementioned implants. Additionally, the system 100 may be implemented according to various methodologies and/or surgical procedures including but not limited to those shown and described herein.

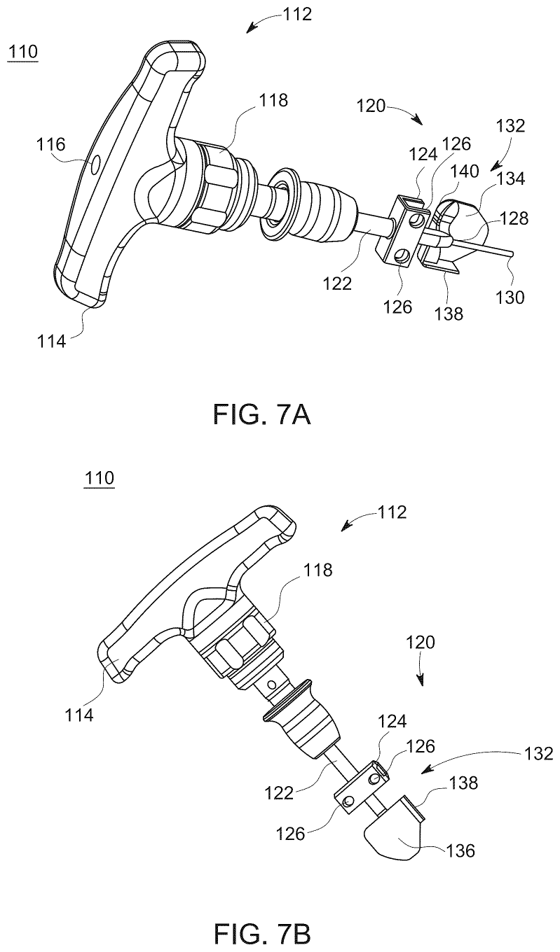

The system 100 includes an actuation instrument 110 (referred to hereinafter as “instrument 110”), which is shown in FIGS. 1-11 as a component implemented in conjunction with other components of the system 100 and independently in FIGS. 7A and 7B. The instrument 100 includes an upper portion 112 configured opposite a lower portion 120. The upper portion 112 includes a handle 114 which, according to an exemplary embodiment, has a substantially t-shaped geometry. However, in some embodiments the handle 114 may have alternate shapes/geometries. The handle 114 is shown to include a cannulation 116 positioned in a central portion thereof and extending longitudinally along a length of the upper portion 112. In some aspects, the cannulation 116 may be sized to receive at least a portion of another component therethrough and/or therein, for example a stabilization wire (which will be referred to hereinafter as “k-wires”). The upper portion 112 further includes an adjustment mechanism 118 positioned between the handle 114 and the lower portion 120, with the adjustment mechanism 118 extending circumferentially around at least a portion of the upper portion 120 (and the cannulation 116 thereof). The adjustment mechanism 118 may be rotated or otherwise actuated in order to facilitate rotation of the handle 114 relative to the other components of the instrument 110 and to retain (e.g., “lock”) the rotational position of the handle 114 relative to the other components of the instrument 110.

The upper portion of the instrument 110 may be releasably couplable with the lower portion 120 of the instrument 110 via a quick-release or other common coupling mechanism. The lower portion 120 is shown to include a shaft 122 including a cannulation 128 which, upon coupling with the upper portion 112, is coaxial and in fluid communication with the cannulation 116 such that the cannulations 116, 128 may collectively receive a k-wire 130 or other component at least partially therein/therethrough. The lower portion 120 also includes a coupling mechanism 124 disposed along a length of the shaft 122 and including a pair of bores 126 positioned on opposite sides of the shaft 122 and configured to receive at least a portion of other components of the system 100 therein to thereby facilitate releasable coupling.

The lower portion 120 also includes a paddle 132 disposed at the distal-most portion of the lower portion 120. As shown, the paddle 132 is integral with the shaft 122, though in some embodiments the paddle 132 may be releasably coupled with the shaft 122 so as to facilitate removal from and rotation/translation/pivoting relative to the shaft 122. The paddle 132 is shown to include a hexagonal shape, though in some aspects the paddle 132 may have alternate geometries. The paddle 132 includes a first surface 134 and a second surface 136, where one or both surfaces may be configured to abut or be positioned adjacent a bone or a resected surface of a bone of a patient when implemented in conjunction with one or more surgical procedures. The first and second surfaces 134, 136 are positioned on opposite sides of the paddle 132 and define planes parallel to one another. Similarly, first and second planes 134, 136 are laterally offset (and thus, parallel) to the longitudinal axis of the cannulation 128 of the lower portion 120. The paddle 132 also includes a limiting feature 138 (e.g., a depth stop, etc.) extending along at least one side of the paddle 132 and having a greater thickness, width, or otherwise lateral dimension than the first and second surfaces 134, 136 (and the portion of the paddle 132 therebetween) of the paddle 132. The limiting feature 138, which is disposed at least partially on a lateral-most portion of the paddle 132, is configured to prevent over insertion over the instrument 110 (and thus, the paddle 132) into an incision of a patient. The limiting feature 138 is further shown to extend distally along at least a portion of the paddle 132 and is adjacent to the first surface 134 so as to provide a reference to a physician as the first surface 134 is positioned against a flat anatomical surface of the patient (e.g., the limiting feature 138 both prevents over insertion of the paddle 132 and provides an overhang configured to facilitate retention of the first surface 134 against a flat, resected bony surface). The limiting feature 138 also includes at least one opening on an upper portion thereof configured to receive a coupling element at least partially therethrough (e.g., a k-wire, etc.) to facilitate releasable coupling of the paddle 132 (and thus, the lower portion 120 and the instrument 110) with the bony anatomy of a patient.

As shown in FIGS. 1-6, a cut guide 142 may be releasably coupled with the paddle 132 of the instrument 110. The cut guide 142 is shown to include a slot 148 and at least one opening 144 adjacent to the slot 148. A fastener 154 (shown in an exemplary embodiment as a screw) may be placed into and through the at least one opening 144 and at least partially into at least one of a pair of openings 140 disposed in an upper portion of the paddle 132. At least one of the openings 140 of the paddle 132 and 144 of the cut guide 142 may include a threading configured to engage a complimentary threading of the fastener 154. Upon coupling, the slot 148 of the cut guide 142 is configured to be positioned parallel (e.g., in a parallel plane) (and spaced at a known distance from) both the first and second surfaces 134, 136 so as to facilitate a cut parallel and at a known distance from the surfaces 134, 136.

The system 100 is shown to include a compression mechanism 150 configured to releasably couple with the lower portion 120 of the instrument 110. The compression mechanism 150 includes a distal end 158 substantially opposite a body 156 from a proximal end 162. A coupling mechanism 152 is shown to protrude (e.g., extend) laterally from the distal end 158 and includes at least one releasable coupling feature, shown in an exemplary embodiment to be a through hole substantially parallel to a longitudinal axis of the compression mechanism 150. The through hole of the coupling mechanism 152 is configured to be aligned with an opening disposed on the lower portion 120 of the instrument 110, for example, one of the openings 124 or 126. A fastener 154, shown in an exemplary embodiment to include a knob and a threaded shaft, is configured to be received through a portion of one of the aforementioned openings, through the entirety of the coupling mechanism 152, and through the remaining portion of the aforementioned opening so as to releasably couple the compression mechanism 150 with the instrument 110. In some aspects, one or more of the openings engaging with the fastener 154 may include threading complimentary to that of the shaft of the fastener 154. However, in some embodiments the fastener 154 and/or openings may include other features (e.g., ball detent, etc.) configured to facilitate coupling and retain the instrument 110 and compression mechanism 150 in a releasably coupled configuration.

The distal end 158 of the compression mechanism 150 is shown to include a pair of bores 160 extending therethrough and configured to receive coupling components (e.g., k-wires) therethrough so as to facilitate releasable coupling with a bone of a patient (for example, a first metatarsal 202). The bores 160 may be parallel to one another, as shown, or in an alternate embodiment may be oblique relative to one another. The body 156 of the compression mechanism 150, which is shown to have a substantially rectangular prism-shaped geometry, includes a cavity 166 disposed in a central portion thereof. As shown, the cavity 166 is in fluid communication with the outside of the body 156 via windows spaced about the body 156 and extending at least partially along a length of each exterior surface of the body 156. The compression mechanism also includes a slider 168 positioned at least partially within the cavity 166. In some aspects, the slider 168, which is shown to have a solid, substantially rectangular prism-shaped geometry, may be shaped so as to have a greater lateral dimension than the windows of the cavity 166 such that the slider 168 is retained within the cavity 166. However, in some aspects the slider 168 may have a lesser lateral dimension than that of the windows of the cavity 166 and may accordingly be positioned within the cavity 166 and removed therefrom.

The slider 168 includes a pair of bores 170 extending therethrough, which may be the same as or similar to the bores 160 of the distal portion 158. Similarly, the bores 170 may be configured to receive coupling components (e.g., k-wires) therein and therethrough so as to facilitate releasable coupling with a bone of a patient (for example, a medial cuneiform 204 as shown). Prior to the insertion of k-wires in the bores 170, the slider 168 may be slid or otherwise manipulated/translated along the length of the cavity 166 until the slider 168 (and thus, the bores 170) are positioned over the bony anatomy with which a physician desires to couple the slider 168. This positioning of the slider 168 may be substantially opposite the cavity 166 from the distal end 158 (e.g., closer to the proximal end 162). The proximal end 162 is shown to include an opening (which may include a threading on an inner surface thereof) disposed on an end portion thereof (substantially coaxial with a longitudinal axis of the compression mechanism 150 and the cavity 166 thereof) configured to receive at least a portion of a biasing element 164 therein. As shown, the biasing element 164 is a knob with a threaded shaft extending therefrom, although in some aspects the biasing element 164 may have alternate geometries. The biasing element 164 and the threaded shaft thereof are configured to engage with the threading of the opening at the proximal end 162 and, upon actuation of the biasing element 164, the shaft enters and extends along the longitudinal axis of the cavity 166. Once a distal-most portion of the biasing element 164 contacts a lateral surface of the slider 166, subsequent actuation of the biasing element 164 repositions at least one of the slider 166 (and subsequently, the bony anatomy with which it is coupled (e.g., the medial cuneiform 204)) and the body 156 of the coupling mechanism 150 (including the distal end 158 and the bony anatomy with which it is coupled, for example the first metatarsal 202) are biased and selectively positioned toward one another in a direction substantially parallel to that of the longitudinal axis of the coupling mechanism 150.

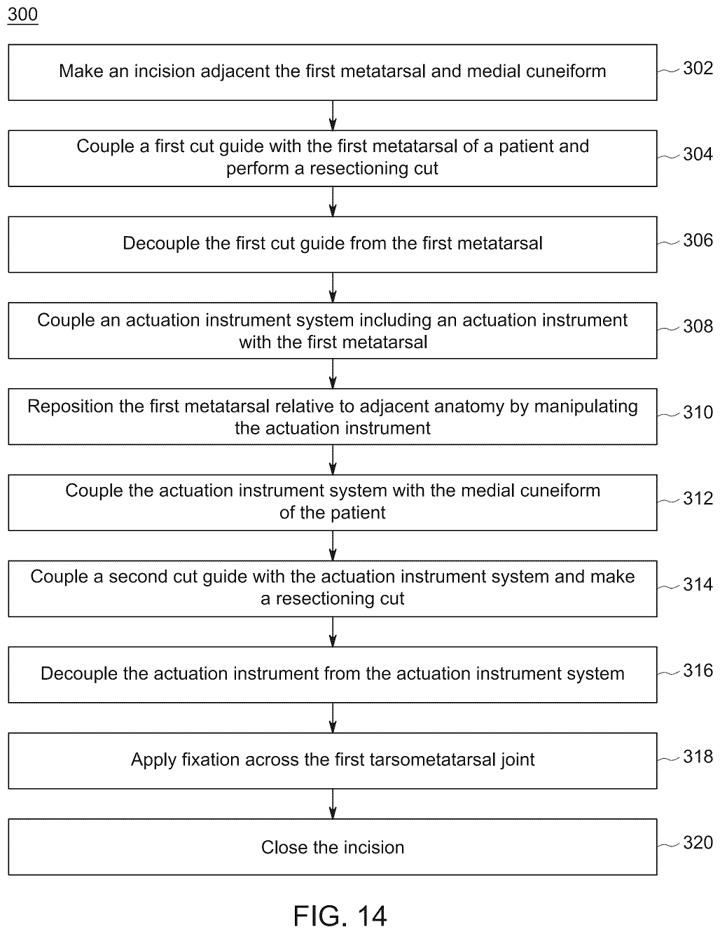

Referring now to FIG. 14, a method or process 300 for performing at least a portion of a bunion procedure is shown, according to an exemplary embodiment. In performing the process 300, the system 100 and/or one or more components thereof (in additional to other systems/components) may be implemented. Further, it should be understood that in performing the process 300, one or more of the steps thereof may be omitted, repeated, performed in an alternate sequence, or replaced with one or more alternate steps.

The process is shown to include a step 302 of making an incision adjacent the first metatarsal and the medial cuneiform, according to an exemplary embodiment. In some aspects, one or more components of the system 100 may be adjacent the anatomy of the patient when a physician performs the step 302.

The process is shown to include a step 304 of coupling a first cut guide with the first metatarsal of a patient and performing a resectioning cut, according to an exemplary embodiment. The step 304 may include implementing a cut guide that is not couplable with other components of the system 100, for example one or more of the cut guides incorporated by reference herein. In some aspects, the cut guide may be coupled with the first metatarsal 202 via a k-wire and the resectioning cut made with a sagittal or reciprocating saw so as to create a flat surface that is not slanted in any of the anterior/posterior/medial/lateral directions.

The process is shown to include a step 306 of decoupling the first cut guide from the first metatarsal 202, according to an exemplary embodiment. As mentioned in step 304, the cut guide may be coupled with the first metatarsal 202 via a k-wire and, accordingly, step 304 may include removing the cut guide over the k-wire rather than removing the k-wire.

The process is shown to include a step 308 of coupling an actuation instrument system including an actuation instrument with the first metatarsal 202, according to an exemplary embodiment. As mentioned with reference to step 306, one or more k-wires placed to couple the cut guide with the first metatarsal 202 may remain in the first metatarsal 202 such that the instrument 110 (which may be coupled with one or more components of the system 100, for example the compression mechanism 150) may be placed over said k-wire such that it is received within the cannulations 116, 128 of the instrument 110. Once the first surface 134 of the paddle 132 is positioned adjacent the resected surface of the first metatarsal 202 (and the limiting feature has guided placement and prevented over insertion of the instrument 110), one or more additional k-wires may be placed (which may be oblique to the initial k-wire) through the openings 160 of the compression mechanism 150.

The process is shown to include a step 310 of repositioning the first metatarsal relative to adjacent anatomy by manipulating the actuation instrument, according to an exemplary embodiment. Step 310 may include manipulating the instrument 110 by grasping the handle and accordingly correcting any deformity of the first metatarsal 202 (which may include one or more of, but is not limited to, rotational and angular deformities).

The process is shown to include a step 312 of coupling the actuation instrument system with the medial cuneiform of the patient, according to an exemplary embodiment. Step 312 may be performed after a physician has determined a corrected position of the first metatarsal 202 and, while retaining the first metatarsal 202 in said position, releasably couples the slider 168 with the medial cuneiform 204 by placing one or more k-wires within the openings 170. When step 312 is performed, the slider 168 may be positioned within the cavity 166 closer to the proximal end 162 than the distal end 164. Further, when step 312 is performed, the fastener 164 may be decoupled from the compression mechanism 150 or coupled such that that shaft portion does not yet or minimally extends into the cavity 166.

The process is shown to include a step 314 of coupling a second cut guide with the actuation instrument system and making a resectioning cut, according to an exemplary embodiment. Step 314 may include releasably coupling the cut guide 142 with the paddle 132 on the instrument 110. In coupling the cut guide 142, the fastener 146 may be placed through at least one of the openings 144 of the cut guide 142 and at least partially into one of the openings 140 of the paddle 132. This coupling may include threadable engagement of one or more portions of one or more of the aforementioned components. Further, step 314 includes ensuring that the slot 148 is positioned in a plane parallel to the second surface 136 of the paddle 132. Accordingly, when the cut is made using an instrument that is the same as or similar to that mentioned previously in the process 300, the resected surface of the medial cuneiform 204 will be in a plane parallel to that of the resected surface of the first metatarsal 202 (and, accordingly, the first and second surfaces 134, 136 of the paddle 132).

The process is shown to include a step 316 of decoupling the actuation instrument from the actuation instrument system, according to an exemplary embodiment. Step 316 may include decoupling the instrument 110 from the compression mechanism 150 via the coupling mechanism 152 and the fastener 154. Further, the k-wire disposed in the cannulations 116, 128 may also be removed in step 316. However, other k-wires placed to couple components of the compression mechanism 150 may remain in place such that the compression mechanism 150 remains coupled with the first metatarsal 202 and the medial cuneiform 204.

The process is shown to include a step 318 of applying fixation across the first tarsometatarsal joint, according to an exemplary embodiment. In some aspects, step 318 may include manipulating the fastener 164 so as to reposition the resected surfaces of the first metatarsal 202 and the medial cuneiform 204 adjacent one another (which may occur by translating one or both of the bony anatomy along an axis that is substantially parallel to that of the compression mechanism 150). Step 318 may be performed prior to or after any/all instrument systems and components of have been decoupled from anatomy of the patient including but not limited to those shown and described herein. For example, the compression mechanism 150 may be removed after fixation has been applied, but the instrument 110 may be decoupled from patient anatomy prior to application of fixation. Step 318 may also include the incorporation of additional hardware (for example, that incorporated by reference previously herein) that is configured to facilitate the application of said fixation. The fixation may include intramedullary nails, bone plates, fasteners, or a combination of several components including but not limited to those mentioned herein.

The process is shown to include a step 320 of closing the incision, according to an exemplary embodiment. Step 320 may include implementing one or more components common to closing surgical incisions, including adhesives and/or stitches/sutures/tapes. Further, this step may be performed before or after all hardware (e.g., instrumentation, systems, etc.) is removed from the patient, as this step may be performed iteratively and, as such, instrument/system/component removal may also occur iteratively.

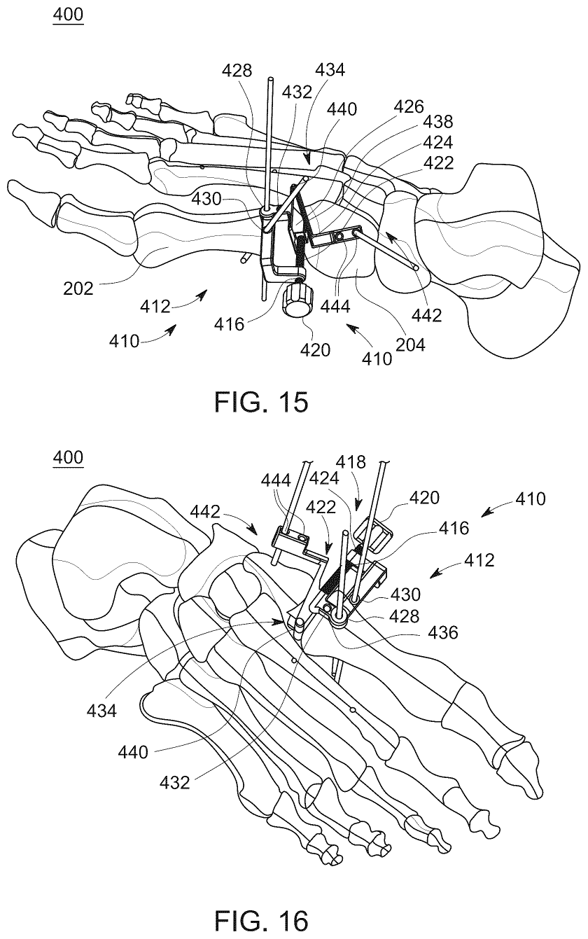

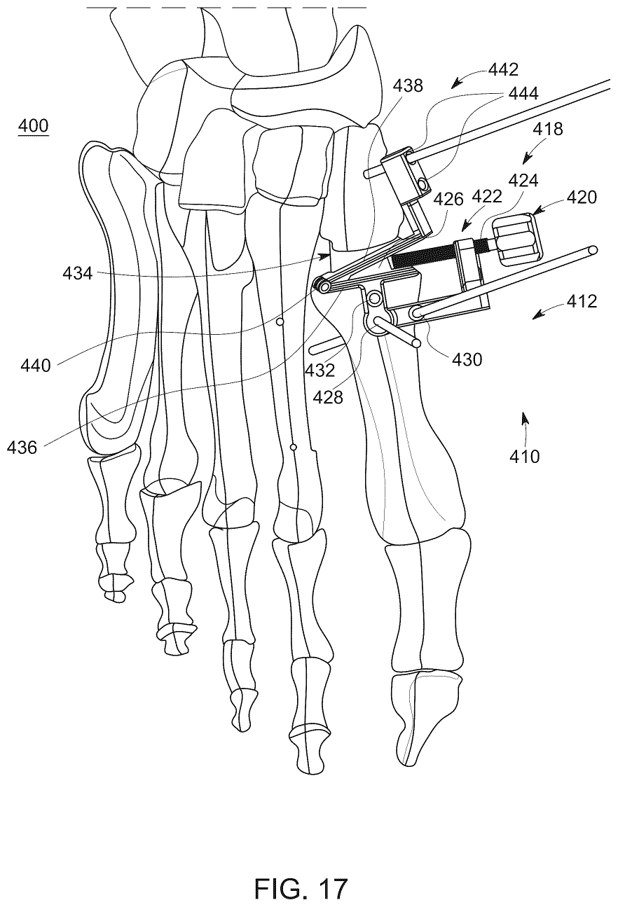

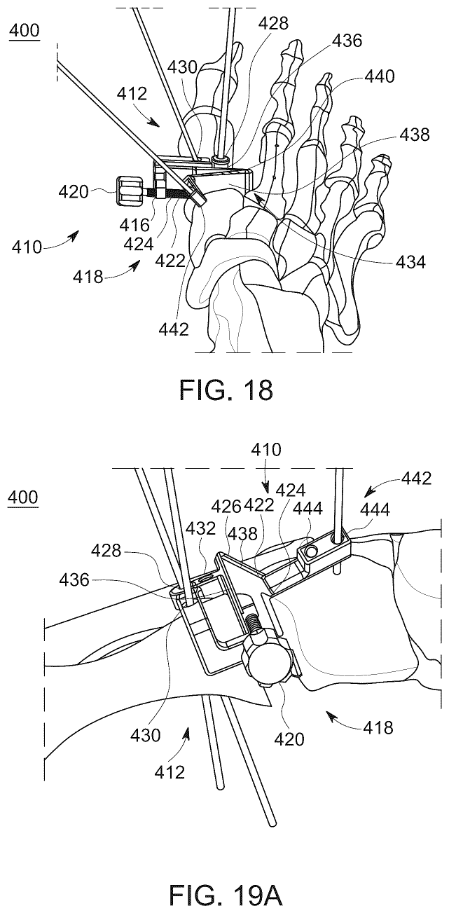

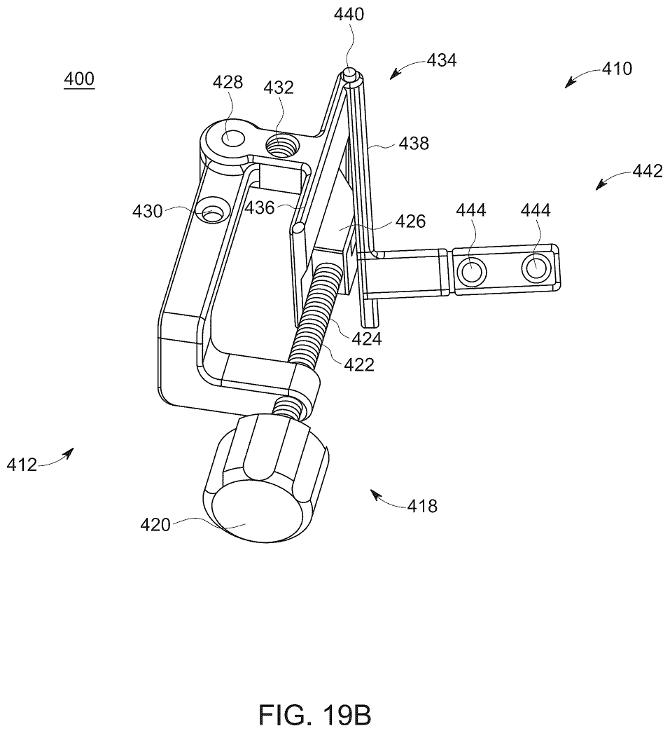

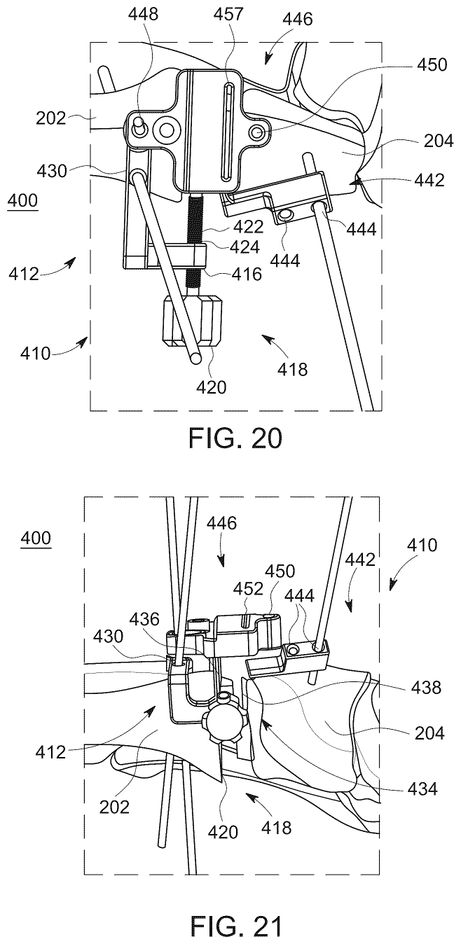

Referring now to FIGS. 15-21, an actuation instrument system 400 (referred to hereinafter as “system 400”) is shown, according to an exemplary embodiment. The system 400 may be implemented in conjunction with various additional instrumentation including those that are common to orthopedic procedures, for example cutting instruments and drills. Further, the system 400 may also be implemented in conjunction with various implant systems and associated instrumentation including, for example, intramedullary nails, bone plates, bone fasteners (e.g., screws, staples, etc.) and instrumentation configured to facilitate the implantation of the aforementioned implants. Additionally, the system 400 may be implemented according to various methodologies and/or surgical procedures including but not limited to those shown and described herein.

The system 400 includes an actuation instrument 410 (referred to hereinafter as “instrument 410”), which is shown in FIGS. 15-21 as a component implemented in conjunction with other components of the system 400. The instrument 410 is shown to include a body portion 412 and a wedge portion 434 positioned substantially adjacent one another. As shown, the body portion 412 and the wedge portion 434 are at least partially integral with one another, although in some aspects the body portion 412 and wedge portion 434 may be releasably couplable with one another. The body portion 412 is shown to include a coupling portion 414 extending (e.g., protruding) substantially laterally from the body portion 412 and including an opening 416 which, as shown, has a longitudinal axis substantially parallel to at least a portion of the body portion 412. The opening 416 may include a threading on at least a portion of an inner surface thereof configured to receive and engage with an actuator 418. The actuator 418 is shown to include a knob 420 and a shaft 422 extending therefrom, with the shaft 422 including a threading 424 disposed on at least a portion of an outer surface thereof. Further, the actuator 418 includes a biasing element 426 disposed at a distal-most portion thereof, extending from the shaft 422. The knob 420 and shaft 422 are integral with one another, but the biasing element 426 is rotatably coupled with the distal portion of the shaft 422 such that rotation of the knob and shaft 420, 422 do not necessarily result in rotation of the biasing element. As shown, the biasing element is substantially wedge shaped and includes a flat surface opposite an angled or radiused substantially curved/arced surface. In some aspects, the biasing element 426 may include alternate geometries. The threading 424 is configured to engage with the aforementioned threading of the opening 416 such that actuation (e.g., rotation) of the actuator 418 drives translation of the actuator 418 (led by the biasing element 416) along the longitudinal axis of the opening 416.

The body portion 412 includes three openings 428, 430, and 432 disposed opposite the body portion 412 from the coupling portion 414 with each configured to receive a coupling element therein (e.g., a k-wire) to facilitate coupling the body portion 414 (and thus, the actuation instrument) with the bony anatomy of the patient (for example, the first metatarsal 202 as shown). The opening 430, positioned closest the coupling portion 414 of the three aforementioned bores, is shown to be an oblique opening, whereas the openings 428 and 432 are not oblique openings and thus have longitudinal axes in planes substantially parallel to that of a longitudinal axis of the body portion 414. The openings 428 and 432 are positioned adjacent one another at an end portion of the body portion 412 opposite the coupling portion 414. As shown, an extension protrudes in the same lateral direction (and having approximately the same length) from the body portion 412 as the coupling portion 414 but at an opposite end thereof, with the opening 432 disposed on said extension and the bore 428 positioned at a corner of the body portion 412 and the extension.

The wedge portion 434 is shown to include a distal paddle 436 and a proximal paddle 438, which are moveably coupled with a hinge 440. The distal paddle 436 as shown is integral with the body portion 412 (specifically, the extension including the opening 430) such that a first surface (e.g., side) of the distal paddle 436 is integral with the body portion 412 while a second, opposite surface of the distal paddle 436 is configured to oppose a first side of the proximal paddle 438. The hinge 440 and associated hinged coupling is arranged opposite the instrument 410 from the coupling portion 414. As shown, the instrument 410 and the aforementioned components thereof are positioned such that the flat surface of the biasing element 426 may contact and translate along/adjacent to the second surface of the distal paddle 436 such that the curved surface of the biasing element 426 contacts the first surface of the proximal paddle 438, thus hingedly separating (or “wedging open”) the distal and proximal paddles 436, 438.

The second surface of the proximal paddle 438 is shown to include an arm 442 that is integral with and extends laterally from the surface. The arm 442 is shown to include a pair of openings 444, which may be the same size as at least one of the openings shown and described previously herein. As shown, the openings 444 are parallel to one another, although in some aspects one or both openings 444 may be angled such that the longitudinal axes of the openings 444 are oblique. Each of the openings 444 are configured to receive a coupling element therethrough, for example a k-wire, so as to facilitate coupling with a bony anatomy (for example, the medial cuneiform 204) positioned beneath the arm 442.

The system 400 is further shown to include a cut guide 446 configured to releasably couple with the medial cuneiform 204 via placement of a k-wire through an opening 450 disposed on the cut guide adjacent a cut slot 452. Further, the cut guide 446 also includes an opening 448 disposed opposite the cut slot 452 from the opening 450 which may receive (e.g., be placed over top of) a k-wire extending upward from the opening 428 of the body portion 412 and coupling the instrument 410 with the first metatarsal 202.

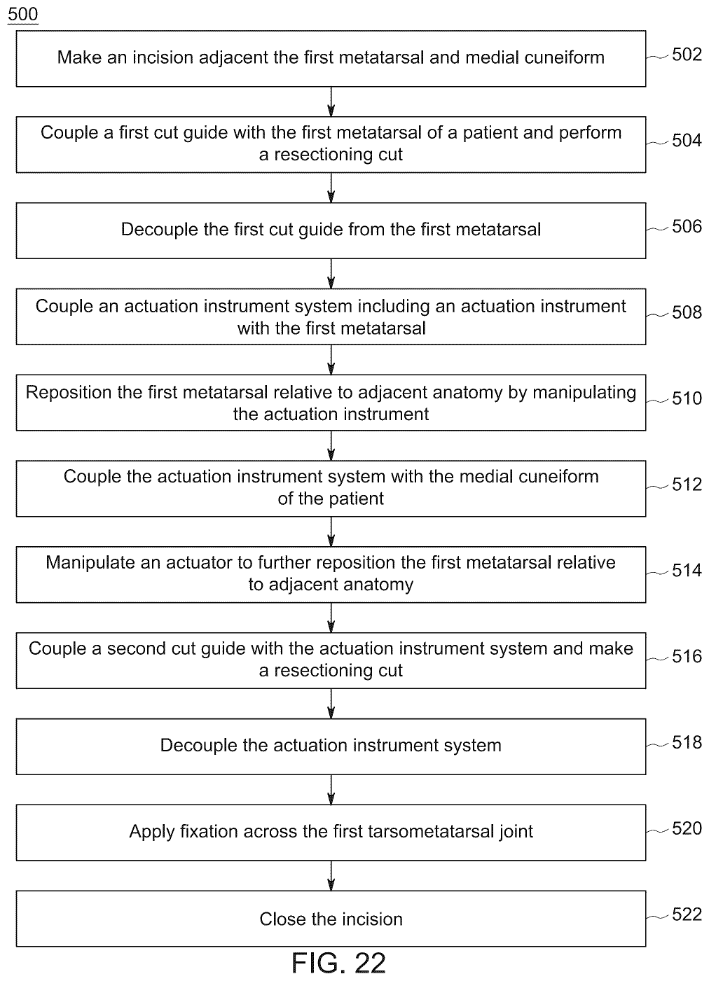

Referring now to FIG. 22, a process or method 500 for performing at least a portion of a bunion procedure is shown, according to an exemplary embodiment. In performing the process 500, the system 400 and/or one or more components thereof (in additional to other systems/components) may be implemented. Further, it should be understood that in performing the process 500, one or more of the steps thereof may be omitted, repeated, performed in an alternate sequence, or replaced with one or more alternate steps.

The process is shown to include step 502 of making an incision adjacent the first metatarsal 202 and the medial cuneiform 204, according to an exemplary embodiment. In some aspects, one or more components of the system 400 may be adjacent to the bony anatomy of the patient when a physician performs step 502.

The process is shown to include step 504 of coupling a first cut guide with the first metatarsal 202 of a patient and performing a resectioning cut, according to an exemplary embodiment. Step 504 may include implementing a cut guide that is not couplable with other components of the system 400, for example one or more of the cut guides incorporated by reference herein. In some aspects, said cut guide may be coupled with the first metatarsal 202 via a k-wire and the resectioning cut made with a sagittal or reciprocating saw so as to create a flat surface that is not slanted in any of the anterior/posterior/medial/lateral directions. In some aspects, step 504 may include approximating a rotational deformity of the first metatarsal 202 of a patient (e.g., 15 degrees from a desired position), and placement of the k-wire at that same approximated rotational measurement from the vertical (in the medial direction) such that restoring the k-wire to vertical would approximately correct the rotational deformity of the first metatarsal 202.

The process is shown to include step 506 of decoupling the first cut guide from the first metatarsal 202, according to an exemplary embodiment. As mentioned in step 504, the cut guide may be coupled with the first metatarsal 202 via a k-wire and, accordingly, step 504 may include removing said cut guide over the k-wire rather than removing the k-wire.

The process is shown to include step 508 of coupling an actuation instrument system including an actuation instrument with the first metatarsal 202, according to an exemplary embodiment. As mentioned with reference to step 506, one or more k-wires placed to couple the cut guide with the first metatarsal 202 may remain in the first metatarsal 202 such that the instrument 410 (which may be coupled with one or more components of the system 400 may be placed over the k-wire such that it is received within the opening 428 of the instrument 410. An additional k-wire may be placed within the opening 432 thus securing the instrument 410 to the first metatarsal 202. Further, step 506 may include positioning at least a portion of the wedge portion 434 between the resected surface of the first metatarsal 202 and the medial cuneiform 204.

The process is shown to include step 510 of repositioning the first metatarsal 202 relative to the adjacent bony anatomy by manipulating the actuation instrument, according to an exemplary embodiment. A portion of the instrument 410, for example the actuator 418, may be grasped and used to rotate the first metatarsal 202 such that the k-wire placed step 506 is restored to vertical.

The process is shown to include step 512 of coupling the actuation instrument system with the medial cuneiform 204 of the patient, according to an exemplary embodiment. Step 512 may be performed after a physician has determined a corrected rotational position of the first metatarsal 202 and, while retaining the first metatarsal 202 in the corrected rotational position, releasably couples the arm 442 with the medial cuneiform 204 by placing one or more k-wires within the openings 444.

The process is shown to include step 514 which is manipulating an actuator to further reposition the first metatarsal 202 relative to the adjacent bony anatomy, according to an exemplary embodiment. Step 514 may include manipulating the actuator 418 via the knob 420 so as to translate the biasing element 426 between the distal and proximal paddles 436, 438 such that the flat surface of the biasing element 426 contacts the second surface of the distal paddle 436 and the curved surface of the biasing element 426 contacts the first surface of the proximal paddle 438, thus “wedging” the two paddles 436, 438 apart as one or both pivots about the hinge 440.

The process is shown to include step 516 of coupling a second cut guide with the actuation instrument system and making a resectioning cut, according to an exemplary embodiment. Step 516 may include releasably coupling the cut guide 446 with the body portion 412 and the k-wire extending upward from the opening 428 such that said k-wire also extends through the opening 448 of the cut guide 446. In coupling the cut guide 446, a k-wire may be placed through the opening 450 so as to couple the cut guide 446 with the medial cuneiform 204. Further, step 516 includes ensuring that the slot 452 is positioned in a plane parallel to the second surface of the distal paddle 436. Accordingly, when the cut is made using an instrument the same as or similar to that mentioned previously in the process 500, the resected surface of the medial cuneiform 204 will be in a plane parallel to that of the resected surface of the first metatarsal 202 (and, accordingly, the first and second surfaces of the distal paddle 436).

The process is shown to include step 518 which is decoupling the actuation instrument from the actuation instrument system, according to an exemplary embodiment. Step 518 may include decoupling the instrument 410 from the anatomy by removing various k-wires.

The process is shown to include step 520 of applying fixation across the first tarsometatarsal joint, according to an exemplary embodiment. Step 520 may be performed prior to or after any/all instrument systems and components of have been decoupled from bony anatomy of the patient including but not limited to those shown and described herein. In some aspects, step 520 may include manipulation of the anatomy that is not dependent on hardware, for example engagement of the Windlass mechanism in order to compress an arthrodesis site (e.g., resected surfaces of the first metatarsal 202 and the medial cuneiform 204). Step 520 may also include the incorporation of additional hardware (for example, that incorporated by reference previously herein) that is configured to facilitate the application of said fixation. The fixation may include intramedullary nails, bone plates, fasteners, or a combination of several components including but not limited to those mentioned herein.

The process is shown to include step 522 of closing the incision, according to an exemplary embodiment. Step 522 may include implementing one or more components common to closing surgical incisions, including adhesives and/or stitches/sutures/tapes. Further, this step may be performed before or after all hardware (e.g., instrumentation, systems, etc.) is removed from the patient, as this step may be performed iteratively and, as such, instrument/system/component removal may also occur iteratively.

It should be understood that the systems 100, 400, and components (as well as the processes 300, 500, and the steps thereof) thereof may be duplicated, replaced, or otherwise modified or manipulated to accommodate various anatomy, implant and instruments systems, and other variables. For example, components of the systems 100, 400 may be offered in larger and smaller sizes than those shown. The systems 100, 400 may also be modified to exclude or provide multiple components shown and described herein. Similarly, the systems 100, 400 may be modified to accommodate implementation in conjunction with other systems including but not limited to implant systems and associated instrumentation. Further, one or more components of the systems 100, 400 may be cross-compatible. For example, components shown and described herein with reference to the system 100 may be implemented in the same or similar capacities in the system 400, and vice-versa.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An actuation instrument, comprising:
an upper portion; and
a lower portion;
wherein the actuation instrument is releasably couplable with at least one of a first metatarsal and an cuneiform of a patient.

2. The instrument of claim 1, wherein the upper portion comprises:
a handle portion; and
a first cannulation, wherein the first cannulation extends along a longitudinal axis of and at least partially through the upper portion.

3. The instrument of claim 2, wherein the lower portion comprises:
a shaft; and
a second cannulation, wherein the second cannulation extends along a longitudinal axis and at least partially through the lower portion.

4. The instrument of claim 3, wherein the upper portion and lower portion of the instrument are releasably couplable with one another.

5. The instrument of claim 4, wherein the lower portion further comprises:
a paddle extending from an end of the shaft opposite that which is releasably couplable with the upper portion, wherein the paddle comprises a pair of planar surfaces configured such that a first plane is positioned parallel to a second plane.

6. The instrument of claim 5, wherein the paddle comprises a greater lateral dimension at a first end positioned adjacent the shaft than at a second end positioned opposite the paddle from the shaft.

7. The instrument of claim 6, wherein the lower portion further comprises:
a coupling mechanism positioned along a length of the shaft and extending laterally therefrom, wherein the coupling mechanism comprises at least one opening extending therethrough from a first side to a second side.

8. The instrument of claim 7, wherein the lower portion further comprises:
a compression mechanism releasably couplable with the coupling mechanism of the shaft.

9. The instrument of claim 8, wherein the compression mechanism is positioned between the paddle and a coupling point between the upper portion and the lower portion when coupled with the coupling mechanism.

10. The instrument of claim 9, wherein the compression mechanism comprises:
a cavity disposed within and extending along a longitudinal axis of the compression mechanism, wherein the cavity provides fluid communication through the compression mechanism from a first end to a second end.

11. The instrument of claim 10, wherein the compression mechanism comprises:
at least one window extending at least partially along a length of the compression mechanism, wherein the at least one window provides fluid communication between outer surfaces of the compression mechanism and the cavity.

12. The instrument of claim 11, wherein the compression mechanism comprises:
a slider disposed and retained within the cavity of the compression mechanism, wherein the slider comprises at least one opening extending therethrough and is translatable within the compression mechanism along at least a portion of the length of the cavity.

13. The instrument of claim 12, wherein the compression mechanism further comprises:
a biasing element comprising a knob and a threaded shaft extending from the knob.

14. The instrument of claim 13, wherein the biasing element is threadably coupled and engaged with the compression mechanism via a threaded opening in fluid communication with the cavity, wherein actuation of the biasing element manipulates at least a portion of the threaded shaft within the cavity so as to bias the slider into a desired position.

15. An instrument system, comprising:
a cut guide; and
an actuation instrument, comprising:
an upper portion comprising a handle and a first cannulation;
a lower portion releasably couplable with the upper portion and comprising:
a shaft;
a second cannulation, wherein the second cannulation is coaxial and in fluid communication with the first cannulation; and
a paddle configured to abut a resected surface of a first bone; and
a compression mechanism releasably couplable with the lower portion, the coupling portion couplable with the first bone and a second bone and comprising:
a biasing element configured to bias at least one of the first bone and the second bone toward each other.

16. The instrument system of claim 15, wherein the compression mechanism further comprises:
a cavity extending along a length of the compression mechanism; and
a slider positioned and retained within the cavity, wherein the slider is translatable along at least a portion of a length of the cavity.

17. The instrument system of claim 16, wherein actuation of the biasing element is configured to translate the slider within the cavity.

18. The instrument system of claim 17, wherein the cut guide is releasably couplable with the lower portion of the actuation instrument.

19. The instrument system of claim 18, wherein the upper portion is releasably couplable with the lower portion of the actuation instrument.

20. A method of using an instrument system to perform a least a portion of a bunion repair procedure, comprising:
making an incision adjacent to a first bone and a second bone;
coupling a first cut guide with the first bone and performing a resection cut;
coupling the first cut guide with the first bone via a k-wire and making a resection cut with a bone saw to create a flat surface;
decoupling the first cut guide from the first bone;
coupling an actuation instrument system including an actuation instrument with the first bone;
repositioning the first bone by manipulating the actuation instrument to correct any deformity of the first bone;
coupling the actuation instrument system with the second bone coupling a second cut guide with the actuation instrument system and making a bone cut through the second bone;
decoupling the actuation instrument from the actuation instrument system;
applying fixation across the space between the first and the second bone and inserting a fastener so as to reposition the resected surfaces of the first bone and the second bone adjacent to one another; and
closing the incision.
